(19) United States
(12) Patent Application Publication
(10) Pub. No.: US 2016/0331959 A1
(43) Pub. Date: Nov. 17, 2016

(54) PROTECTIVE EQUIPMENT HAVING THERMOTHERAPY AND ELECTROTHERAPY FUNCTIONS

(71) Applicant: Well-Life Healthcare Limited, New Taipei City (TW)

(72) Inventor: Chin-Chih HSIEH, New Taipei City (TW)

(21) Appl. No.: 14/708,512

(22) Filed: May 11, 2015

(51) Int. Cl. A61N 1/08; A61F 7/02; A61N 1/36

(52) U.S. Cl. CPC A61N 1/08; A61N 1/36014; A61F 7/02

(57) ABSTRACT

A protective equipment having thermotherapy and electrotherapy functions includes a main body and a control mainframe; the main body includes an inner layer, an outer layer, a heating layer and two electrode layers. The heating layer is disposed between the inner layer and the outer layer, and the two electrode layers are fastened in the inner layer; and the control mainframe is detachably installed on the main body and respectively and electrically connected to the heating layer and the two electrode layers. Accordingly, the main body includes the heating layer and the electrode layers thereby enabling the protective equipment to have both thermotherapy and electrotherapy functions and allowing the operation convenience of the protective equipment to be increased.

### BACKGROUND OF THE INVENTION

1. Field of the Invention

The present invention relates to a protective equipment used in a medical field, especially to a protective equipment having thermotherapy and electrotherapy functions

2. Description of Related Art

When a human being keeps himself/herself in a certain posture for a long period of time, his/her shoulders, neck or waist may feel stiff and uncomfortable, so a thermotherapy or an electrotherapy could be one of the suitable options for relief. As such, a hot pressing belt and a low-frequency electrical stimulator are available in the market for providing a function of relaxing muscles.

However, if a user wants both of the thermotherapy and electrotherapy treatments, he/she has to respectively purchase a hot compressing belt and an electrical stimulator, and the time required for the thermotherapy and electrotherapy treatment may not be the same, so disadvantages such as spending unnecessary money and wasting too much time are caused.

Accordingly, the applicant of the present invention has devoted himself for improving the mentioned disadvantages.

### SUMMARY OF THE INVENTION

The present invention is to provide a protective equipment having thermotherapy and electrotherapy functions, in which a main body thereof includes a heating layer and two electrode layers, thereby enabling the protective equipment to be provided with both of the thermotherapy and electrotherapy functions and allowing the operation convenience of the protective equipment to be increased.

Accordingly, the present invention provides a protective equipment having thermotherapy and electrotherapy functions, which includes: a main body, including an inner layer, an outer layer, at least one heating layer and at least two electrode layers, wherein the heating layer is disposed between the inner layer and the outer layer, and the two electrode layers are fastened in the inner layer; and a control mainframe, detachably installed on the main body and respectively and electrically connected to the heating layer and the two electrode layers.

Accordingly, the present invention provides a protective equipment having thermotherapy and electrotherapy functions, which includes: a main body, including an inner layer, an outer layer, at least one heating layer and at least two electrode layers, wherein the heating layer is disposed between the inner layer and the outer layer, and the two electrode layers are fastened in the inner layer.

In comparison with prior art, advantages achieved by the present invention are as follows:

First, the electrotherapy function provided by the protective equipment can be selective for being wired or wireless, thereby expanding the application of the protective equipment and allowing the protective equipment to have an advantage of convenience in use.

Second, only when a switch member and a signal emitting unit are both actuated, a circuit board is enabled to conduct a current through a battery so as to be transferred to each buckle part, thereby avoiding accidentally triggering the switch and allowing the operation safety of the protective equipment to be enhanced.

Third, the signal transmission of the protective equipment can be changed from a wired means to a wireless mean, so the cost for wires is saved, a situation of the wires limiting the operation range and angle of the protective equipment can be prevented, and a situation of wires being twisted with each other can also be avoided, thereby allowing the protective equipment to be provided with advantages of easy in operation and convenient in storage.

### BRIEF DESCRIPTION OF DRAWING

FIG. 1 is a perspective view showing the assembly of the protective equipment according to a first embodiment of the present invention;

FIG. 2 is a perspective view showing the assembly of the protective equipment according to a second embodiment of the present invention;

FIG. 3 is a cross sectional view showing the protective equipment according to the second embodiment of the present invention; and

FIG. 4 is a perspective view showing the assembly of the protective equipment according to a third embodiment of the present invention.

### DETAILED DESCRIPTION OF THE INVENTION

Preferred embodiments of the present invention will be described with reference to the drawings.

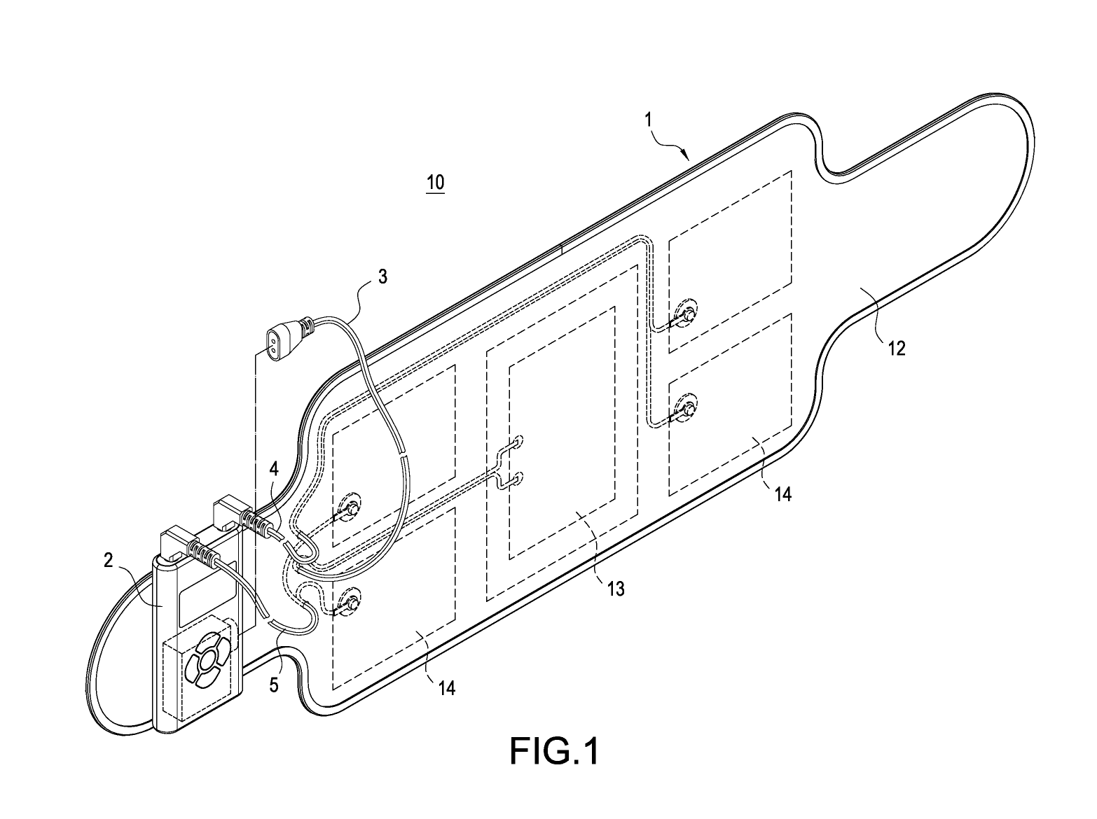

Please refer to FIG. 1, the present invention provides a protective equipment having thermotherapy and electrotherapy functions, wherein the protective equipment 10 mainly includes a main body 1 and a control mainframe 2.

The main body 1 includes an inner layer 11, an outer layer 12, one or a plurality of heating layers 13 and two or multiples of two electrode layers 14, wherein the heating layer 13 is disposed between the inner layer 11 and the outer layer 12, and the two electrode layers 14 are fastened in the inner layer 1.

The control mainframe 2 is detachably installed on the main body 1 and respectively and electrically connected to the heating layer 13 and the two electrode layers 14, and the control mainframe 2 is utilized for controlling the protective equipment 10 to actuate or stop the functions of thermotherapy (for example hot compressing) or electrotherapy.

The protective equipment 10 of the present invention further includes a first wire 3, a second anode wire 4 and a second cathode wire 5, wherein the first wire 3, the second anode wire 4 and the second cathode wire 5 are respectively arranged between the inner layer 11 and the outer layer 12; one end of the first wire 3 is fastened with the heating layer 13 and the other end thereof is detachably inserted in the control mainframe 2, one end of the second anode wire 4 is fastened with one of the electrode layers 14 and the other end thereof is detachably inserted in the control mainframe 2, and one end of the second cathode wire 5 is fastened with the other electrode layer 14 and the other end thereof is detachably inserted in the control mainframe 2.

The assembly of the protective equipment 10 provided by the present invention is that: the main body 1 includes the inner layer 11, the outer layer 12, the heating layer 13 and the two electrode layers 14, the heating layer 13 is disposed between the inner layer 11 and the other layer 12, the two electrode layers 14 are fastened in the inner layer 11; the control mainframe 2 is detachably installed on the main body 1 and respectively and electrically connected to the heating layer 13 and the two electrode layers 14. Accordingly, the protective equipment 10 is provided with both functions of thermotherapy (for example hot compressing) and electrotherapy, thereby greatly increasing the operation convenience of the protective equipment 10.

The operating status of the protective equipment 10 provided by the present invention is that: because the main body 1 includes the heating layer 13 and the electrode layers 14, and the control mainframe 2 is respectively and electrically connected to the heating layer 13 and the two electrode layers 14, the protective equipment 10 is provided with both functions of thermotherapy (for example hot compressing) and electrotherapy, thus a user can enjoy thermotherapy and electrotherapy at the same time and does not have to spend money for respectively buying a heat compressing belt and an electrical stimulator, so the protective equipment 10 of the present invention is provided with advantages of saving time and expenditure.

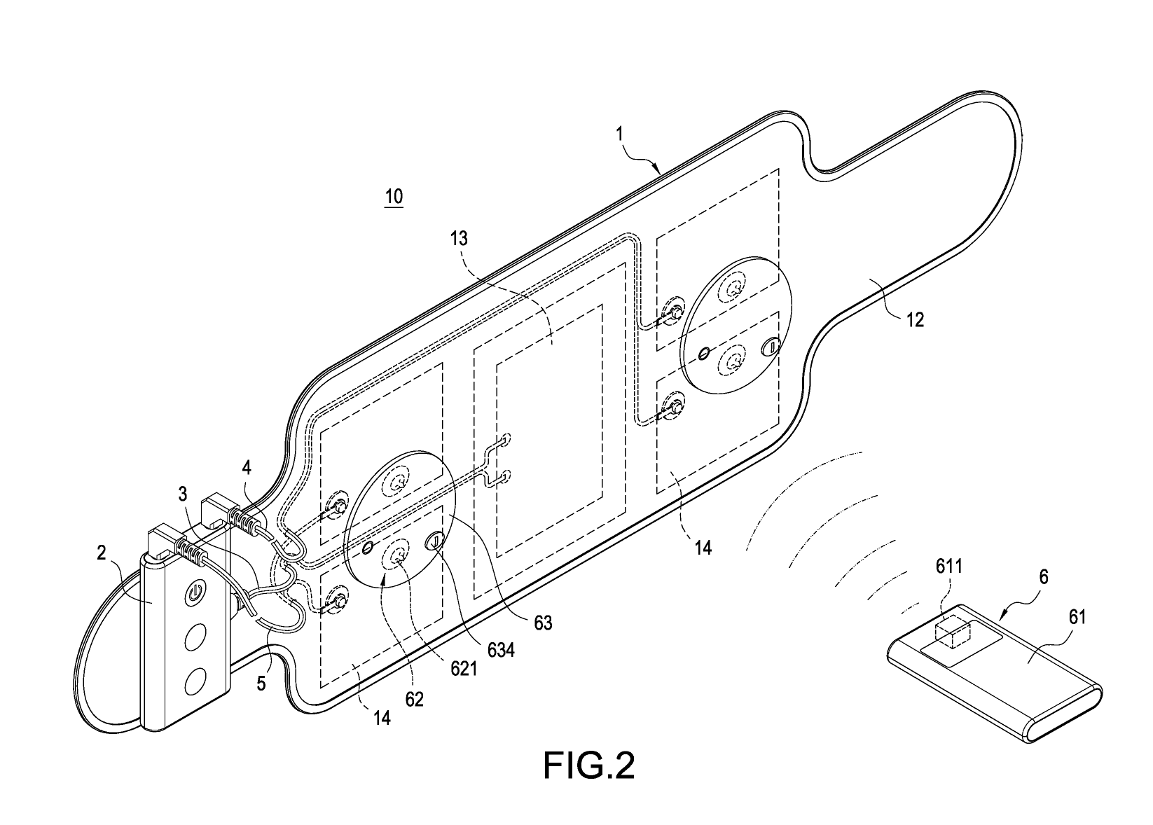

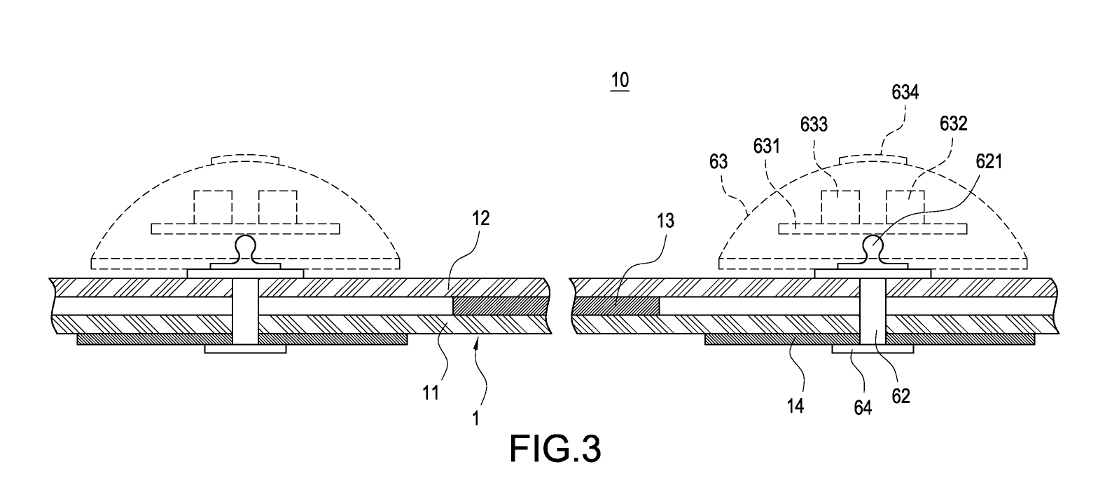

Please refer to FIG. 2 and FIG. 3 which disclose a second embodiment of the protective equipment 10 provided by the present invention, the second embodiment is substantially the same as the first embodiment, and the difference between the second embodiment and the first embodiment is that the protective equipment 10 further includes a wireless electrotherapy device 6.

The wireless electrotherapy device 6 includes a wireless mainframe 61, two or multiples of two electrode buttons 62, two or multiples of two electric buckle units 63 and two or multiples of two adhesive pads 64.

Details are provided as follows. The wireless mainframe 61 includes a signal emitting unit 611, each of the electrode buttons 62 is throughoutly installed in the main body 1 and electrically connected to each of the electrode layers 14, and each of the electrode buttons 62 is formed with a buckle part 621 exposed outside the outer layer 12, each of the electric buckle units 63 is correspondingly buckled with each of buckle parts 621, the interior of each of the electric buckle units 63 includes a circuit board 631, a battery 632 and a wireless receiving module 633, when the wireless receiving module 633 receives an electric signal generated by the signal emitting unit 611, the circuit board 631 is served to electrically conduct a current through the battery 632 so as to be transferred to each of the buckle parts 621, and each of the adhesive pads 64 is adhered corresponding to the inner layer 11 and served to cover each of the electrode buttons 62. Accordingly, the electrotherapy function provided by the protective equipment 10 can be selective for being wired or wireless, thereby expanding the application of the protective equipment 10 and allowing the protective equipment 10 to have an advantage of convenience in use.

In addition, the electric buckle unit 63 further includes a switch member 634 electrically connected to the circuit board 631, the switch member 634 is fastened and exposed outside the electric buckle unit 63, so only when the switch member 634 and the signal emitting unit 611 are both actuated, the circuit board 631 is enabled to conduct a current through the battery 632 so as to be transferred to each of the buckle parts 621, thereby avoiding accidentally triggering the switch and allowing the operation safety of the protective equipment 10 to be enhanced.

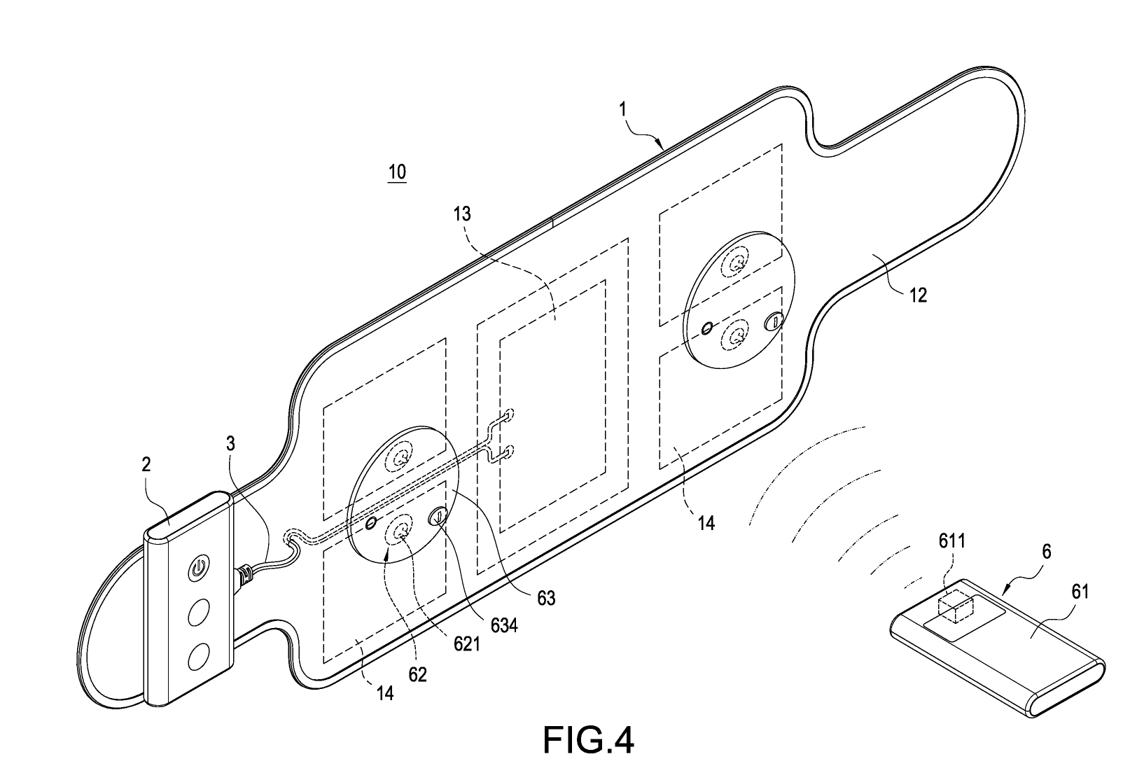

Please refer to FIG. 4, which discloses a third embodiment of the protective equipment 10 provided by the present invention, the third embodiment is substantially the same as the second embodiment, and the difference between the third embodiment and the second embodiment is that the electrotherapy function of the protective equipment 10 is only achieved by a wireless means and a wired means is not adopted.

Details are provided as follows. According to the protective equipment 10 disclosed in this embodiment, the wires used for electrically connecting the control mainframe 2 and the electrode layers 14 are not installed, so the control mainframe 2 is only served to control the thermotherapy operation, and the electrotherapy function of the protective equipment 10 is controlled by the wireless mainframe 61 thereby saving the installation of the required wires, so the cost for wires is saved, a situation of the wires limiting the operation range and angle of the protective equipment 10 can be prevented, and a situation of wires being twisted with each other can also be avoided, thereby allowing the protective equipment 10 to be provided with advantages of easy in operation and convenient in storage.

Although the present invention has been described with reference to the foregoing preferred embodiment, it will be understood that the invention is not limited to the details thereof. Various equivalent variations and modifications can still occur to those skilled in this art in view of the teachings of the present invention. Thus, all such variations and equivalent modifications are also embraced within the scope of the invention as defined in the appended claims.

What is claimed is:

1. A protective equipment having thermotherapy and electrotherapy functions, including:
a main body, including an inner layer, an outer layer, at least one heating layer and at least two electrode layers, wherein the heating layer is disposed between the inner layer and the outer layer, and the two electrode layers is fastened in the inner layer; and
a control mainframe, detachably installed on the main body and respectively and electrically connected to the heating layer and the two electrode layers.

2. The protective equipment having thermotherapy and electrotherapy functions according to claim 1, further including a first wire, a second anode wire and a second cathode wire, wherein the first wire, the second anode wire and the second cathode wire are respectively arranged between the inner layer and the outer layer; one end of the first wire is fastened with the heating layer and the other end thereof is detachably inserted in the control mainframe, one end of the second anode wire is fastened with one of the electrode layers and the other end thereof is detachably inserted in the control mainframe, and one end of the second cathode wire is fastened with the other electrode layer and the other end thereof is detachably inserted in the control mainframe.

3. The protective equipment having thermotherapy and electrotherapy functions according to claim 1, further including a wireless electrotherapy device, wherein the wireless electrotherapy device includes a wireless mainframe, at least two electrode buttons, at least two electric buckle units and at least two adhesive pads; the wireless mainframe includes a signal emitting unit, each of the electrode buttons is throughoutly installed in the main body and electrically connected to each of the electrode layers, and each of the electrode buttons is formed with a buckle part exposed outside the outer layer, each of the electric buckle units is correspondingly buckled with each of the buckle parts, the interior of each of the electric buckle units includes a circuit board, a battery and a wireless receiving module; when the wireless receiving module receives an electric signal generated by the signal emitting unit, the circuit board is served to electrically conduct a current through the battery so as to be transferred to each of the buckle parts, and each of the adhesive pads is adhered corresponding to the inner layer and served to cover each of the electrode buttons.

4. The protective equipment having thermotherapy and electrotherapy functions according to claim 3, wherein the electric buckle unit further includes a switch member electrically connected to the circuit board, and the switch member is fastened and exposed outside the electric buckle unit.

5. A protective equipment having thermotherapy and electrotherapy functions, including:
a main body, including an inner layer, an outer layer, at least one heating layer and at least two electrode layers, wherein the heating layer is disposed between the inner layer and the outer layer, and the two electrode layers is fastened in the inner layer.

6. The protective equipment having thermotherapy and electrotherapy functions according to claim 5, further including a control mainframe and a first wire, wherein the control mainframe is detachable installed on the main body, the first wire is disposed between the inner layer and the outer layer, and one end of the first wire is fastened with the heating layer and the other end thereof is detachably inserted in the control mainframe.

7. The protective equipment having thermotherapy and electrotherapy functions according to claim 5, further including a wireless electrotherapy device, wherein the wireless electrotherapy device includes a wireless mainframe, at least two electrode buttons, at least two electric buckle units and at least two adhesive pads; the wireless mainframe includes a signal emitting unit, each of the electrode buttons is throughoutly installed in the main body and electrically connected to each of the electrode layers, and each of the electrode buttons is formed with a buckle part exposed outside the outer layer, each of the electric buckle units is correspondingly buckled with each of the buckle parts, the interior of each of the electric buckle units includes a circuit board, a battery and a wireless receiving module; when the wireless receiving module receives an electric signal generated by the signal emitting unit, the circuit board is served to electrically conduct a current through the battery so as to be transferred to each of the buckle parts, and each of the adhesive pads is adhered corresponding to the inner layer and served to cover each of the electrode buttons.

8. The protective equipment having thermotherapy and electrotherapy functions according to claim 7, wherein the electric buckle unit further includes a switch member electrically connected to the circuit board, and the switch member is fastened and exposed outside the electric buckle unit.

9. The protective equipment having thermotherapy and electrotherapy functions according to claim 5, further including a control mainframe, a second anode wire and a second cathode wire, wherein the control mainframe is detachably installed on the main body, the second anode wire and the second cathode wire are respectively arranged between the inner layer and the outer layer; one end of the second anode wire is fastened with one of the electrode layers and the other end thereof is detachably inserted in the control mainframe, and one end of the second cathode wire is fastened with the other electrode layer and the other end thereof is detachably inserted in the control mainframe.

10. The protective equipment having thermotherapy and electrotherapy functions according to claim 9, further including a wireless electrotherapy device, wherein the wireless electrotherapy device includes a wireless mainframe, at least two electrode buttons, at least two electric buckle units and at least two adhesive pads; the wireless mainframe includes a signal emitting unit, each of the electrode buttons is throughoutly installed in the main body and electrically connected to each of the electrode layers, and each of the electrode buttons is formed with a buckle part exposed outside the outer layer, each of the electric buckle units is correspondingly buckled with each of the buckle parts, the interior of each of the electric buckle units includes a circuit board, a battery and a wireless receiving module; when the wireless receiving module receives an electric signal generated by the signal emitting unit, the circuit board is served to electrically conduct a current through the battery so as to be transferred to each of the buckle parts, and each of the adhesive pads is adhered corresponding to the inner layer and served to cover each of the electrode buttons.

11. The protective equipment having thermotherapy and electrotherapy functions according to claim 10, wherein the electric buckle unit further includes a switch member electrically connected to the circuit board, and the switch member is fastened and exposed outside the electric buckle unit.
